(19) United States
(12) Patent Application Publication
(10) Pub. No.: US 2025/0382397 A1
(43) Pub. Date: Dec. 18, 2025

(54) BOTTLEBRUSH DELIVERY SYSTEMS AND USES THEREOF

(71) Applicant: Regents of the University of Minnesota, Minneapolis, MN (US)

(72) Inventors: Theresa REINEKE, Vadnais Heights, MN (US); Rishad DALAL, Minneapolis, MN (US)

(21) Appl. No.: 18/880,015

(22) PCT Filed: Jun. 27, 2023
(86) PCT No.: PCT/US2023/026340

§ 371 Date: Dec. 30, 2024

Related U.S. Application Data
(60) Provisional application No. 63/356,847, filed on Jun. 29, 2022.

(51) Int. Cl. C08F 26/06; C12N 9/22; C12N 15/11; C12N 15/88

(52) U.S. Cl. CPC C08F 26/06; C12N 9/226; C12N 15/11; C12N 15/88; C12N 2310/20

(57) ABSTRACT

This invention relates generally to Bottlebrush polymeric delivery systems. The present polymeric delivery systems may be complexed with biological agents, including nucleic acids, peptides, proteins, or small molecules, for delivery to cells. In particular, the present polymeric delivery systems may be used for delivery of Cas protein in gene editing.

### BACKGROUND OF THE INVENTION

Delivery of biological agents, such as nucleic acids, peptides, proteins, or small molecules, to cells both in vitro and in vivo has been performed using various recombinant viral vectors, lipid delivery systems, and electroporation. Such techniques have sought to treat various diseases and disorders by reducing or inhibiting gene expression, providing genetic constructs for gene therapy or to study various biological systems.

Genome editing, for example, based on clustered regularly interspersed palindromic repeats (CRISPR) technology has transformed the therapeutic landscape for diseases wherein the deletion, insertion or repair of genetic sequences can restore healthy cellular states. Clinical trials of investigational gene therapeutics for β-thalassemia and sickle cell disease suggest that safe and efficacious treatment is possible using CRISPR-based genome editing technology. Additional clinical trials are underway to develop CRISPR-based therapeutics for debilitating conditions such as Duchenne's muscular dystrophy (DMD), Leber congenital amaurosis (LCA) and for chimeric antigen receptor T-cell (CAR-T) therapies for cancer.

Despite the vast curative potential of CRISPR, widespread clinical deployment faces an uncertain outlook due to reliance on engineered viral vectors, which can be used to deliver therapeutic biomacromolecule payloads such as messenger RNA (mRNA), plasmid DNA (pDNA) and small interfering RNA (siRNA). However, the high costs, lengthy time requirements, and regulatory challenges involved in manufacturing clinical grade viruses at scale for large patient populations have imposed severe logistical bottlenecks. In addition to manufacturing and regulatory delays, the cargo capacity of viral vectors is limited, and this size restriction is particularly problematic in the context of bulky multi-component CRISPR cargoes.

Although advances in virus manufacturing have minimized occurrences of carcinogenic mutations, genomic integration and fatal systemic inflammatory responses, these risks are amplified when repeated dosing or large dosages are involved. For CRISPR therapeutics to become safe, scalable, and affordable, there is a need to identify synthetic substitutes for viral carriers.

Polymeric delivery vehicles have been used in clinical therapies due to their versatility, relative low production cost, and low immunogenicity. Synthetic polymers have been used to deliver biomacromolecule payloads such as, for example, pDNA, ribonucleoproteins (RNP), and the like, due to their versatility, low toxicity, and the ability to encapsulate large payloads. Some recent examples indicate that synthetic polymer-based systems achieved biomacromolecule based gene delivery and gene editing both in vitro and in vivo.

For example, in aqueous physiological solutions, cationic polymers can spontaneously bind with negatively charged pDNA and form interpolyelectrolyte complexes. These complexes are predominately internalized by various endocytic routes, followed by cargo release from these vesicles inside the cells via different proposed mechanisms and subsequent entry into the cell nucleus to promote gene expression. Compared to viral vehicles, polymeric delivery systems typically have lower delivery efficiency, and various optimization strategies can be used to improve this parameter such as changing the cationic moieties on polymers, adding targeting ligands, and installing responsive monomers, which can improve uptake efficiency and help to balance transfection efficiency and cytotoxicity. However, their utility in genome editing is relatively underexplored.

Novel and efficient polymer-based delivery vehicles are thus desired.

### SUMMARY OF THE INVENTION

The present invention is related to polymeric delivery systems. The present polymeric delivery systems may be complexed with biological agents, including nucleic acids, peptides, proteins, or small-molecules, for delivery to cells.

In one aspect, the invention features a polymer of formula (I)

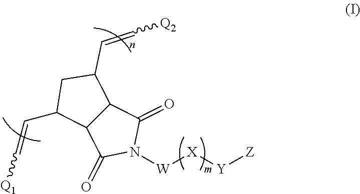

wherein W is alkyl alkanoate; X is a monomeric unit of aminoalkyl acrylate; Y is a bond or S; Z is hydrogen, alkyl,

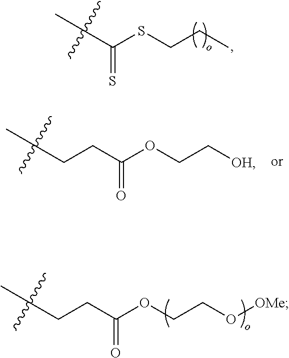

Q₁ is hydrogen or an end group; Q₂ is hydrogen or an end group; n is from 1 to 1000; m is from 1 to 1000; and o is from 1 to 20; or an ion or salt thereof.

In some embodiments, the aminoalkyl acrylate is 2-(dimethylamino)ethyl methacrylate (DMAEMA).

In some embodiments, n is from about 5 to about 100. In some embodiments, n is from about 10 to about 50, e.g., about 30. In some embodiments, m is from about 5 to about 100. In some embodiments, m is from about 10 to about 50, e.g., about 20.

In some embodiments, Q₁ or Q₂ is an end group, e.g., alkyl, aryl, or heterocyclyl. In some embodiments, Q₁ is phenyl. In some embodiments, Q₂ is hydrogen. In some embodiments, Q₁ is phenyl, and Q₂ is hydrogen.

In another aspect, the invention features a complex including a polymer of any one of the presently described polymers and a negatively charged biological agent.

In some embodiments, the negatively charged biological agent includes a nucleic acid. In some embodiments, the nucleic acid includes DNA or RNA. In some embodiments, the nucleic acid includes gRNA, mRNA, tmRNA, tRNA, rRNA, siRNA, shRNA, PNA, ssRNA, dsRNA, pDNA, ssDNA, dsDNA, a DNA:RNA hybrid molecule, a plasmid, an artificial chromosome, cDNA, a PCR product, a restriction fragment, a ribozyme, an antisense construct, or a combination thereof.

In some embodiments, the negatively charged biological agent includes a protein. In some embodiments, the protein includes a ribonucleoprotein. In some embodiments, the ribonucleoprotein includes a virus, a ribosome, telomerase, Ribonuclease P (RNase P), a heterogeneous ribonucleoprotein particle (hnRNP), or a small nuclear ribonucleoprotein particle (snRNP). In some embodiments, the protein includes a nuclease. In some embodiments, the nuclease includes a zinc finger nuclease (ZFNs), a transcription-activator like effector nucleases (TALEN), or a Cas protein. In some embodiments, the Cas protein includes Cas2, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas12, Cas13, Cas14, CfpI, CasI, CasIB, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, modified versions thereof, or combinations thereof. In some embodiments, the Cas protein is Cas9.

In some embodiments, the negatively charged biological agent includes a nucleic acid and a nuclease. In some embodiments, the negatively charged biological agent includes gRNA and a Cas protein.

In some embodiments, the negatively charged biological agent is bound noncovalently to the polymer. In some embodiments, the polymer is complexed with the negatively charged biological agent.

In another aspect, the invention features a composition including a complex of any one of the presently described complexes and a liquid carrier. In another aspect, the invention features a method including contacting a cell with a complex of any one of the presently described complexes, wherein the biological agent is delivered into the cell.

In another aspect, the invention features a compound of formula (II)

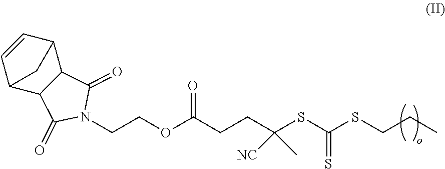

wherein o is from 1 to 20.

In another aspect, the invention features a polymer of formula (III)

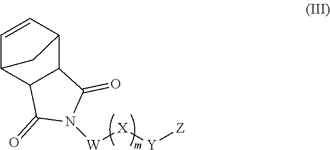

wherein W is alkyl alkanoate; X is a monomeric unit of aminoalkyl acrylate; Y is a bond or S; Z is hydrogen, alkyl,

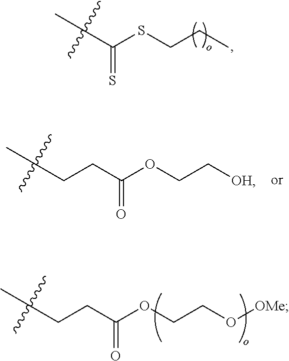

m is from 1 to 1000; and o is from 1 to 20; or an ion or salt thereof.

### Definitions

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

Terms such as “a”, “an,” and “the” are not intended to refer to only a singular entity but include the general class of which a specific example may be used for illustration.

The term “about,” as used herein, refers to a value that is within 10% above or below the value being described.

The term “acrylate,” as used herein, refers to a compound of formula:

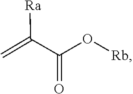

where Ra is H or alkyl, e.g., methyl, and Rb is alkyl, e.g., aminoalkyl. A monomeric unit of acrylate has the formula:

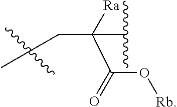

The term “alkyl alkanoate,” as used herein, refers to a divalent moiety of the formula —R¹C(═O)OR²—, wherein R¹ and R² are alkylene groups. The group may be attached in either orientation.

The term “aminoalkyl,” as used herein, refers to an (NR3R⁴) R⁵-group, wherein R³ and R⁴ are independently H, alkyl, or cycloalkyl, and R⁵ is alkyl.

The term “alkyl,” as used herein, refers to an acyclic straight or branched chain, saturated, monovalent hydrocarbon group having from 1 to 12 carbons (e.g., 1 to 6), unless otherwise specified. Alkyl groups may be substituted or unsubstituted. Exemplary substituents include alkoxy, alkylthio, amido, amino, carbonate, carboxyl, cyano, epoxy, halo, heterocyclyl, hydroxyl, oxo, and thiol. An alkyl may be substituted with an oxo to form an aldehyde or ketone.

The term “alkoxy,” as used herein, refers to a group of the formula RO—, wherein R is an alkyl group as defined herein. Alkoxy groups may be unsubstituted or substituted as alkyl groups. An alkoxy may be substituted with an oxo group to form an ester. Three alkoxy groups may be bound to the same carbon to form an orthoester.

The term “alkylthio,” as used herein, refers to a group of the formula RS—, wherein R is an alkyl group as defined herein. Alkylthio groups may be unsubstituted or substituted as alkyl groups.

The term “alkylene,” as used herein, refers to a divalent group obtained by removing a hydrogen from a carbon atom of an alkyl group. Alkylene groups may be unsubstituted or substituted as alkyl groups.

The term “amido,” as used herein, refers to a group of the formula —C(═O) NR′R″, where each of R′ and R″ are independently H or alkyl.

The term “amino,” as used herein, refers to a group of formula —NR′R″ or —NR′R″R′″+, where each of R′, R″, and R′″ are independently H or alkyl.

The term “aryl,” as used herein, refers to any monocyclic or fused ring bicyclic or multicyclic system containing only carbon atoms in the ring(s), which has the characteristics of aromaticity in terms of electron distribution throughout the ring system, e.g., phenyl, naphthyl, or phenanthryl. An aryl group may have, e.g., six to sixteen carbons (e.g., six carbons, ten carbons, thirteen carbons, fourteen carbons, or sixteen carbons). Aryl groups may be unsubstituted or substituted. Exemplary substituents include alkyl, alkoxy, alkylthio, amido, amino, aryl, carbonate, carboxyl, cyano, epoxy, halo, heterocyclyl, hydroxyl, and thiol.

The term “carbonate,” as used herein, refers to a group of the formula —OC(═O)OR, wherein R is H or alkyl.

The term “carboxyl,” as used herein, refers to a group of the formula —(C═O) OH.

The term “cyano,” as used herein, refers to —C≡N.

The term “cycloalkyl,” as used herein, refers to a cyclic saturated hydrocarbon group having from 3 to 12 carbons (e.g., 3-6), unless otherwise specified.

The term “effective amount,” as used herein refers to the amount that is necessary to result in a physiological change in the cell, organism, or tissue to which it is administered.

The term “epoxy,” as used herein, refers to >O, where the oxygen is bound to adjacent carbon atoms.

The term “halide,” as used herein, refers to a F, Cl, Br, or I anion.

The term “halo,” as used herein, refers to a F, Cl, Br, or I radical.

The term “heterocyclyl,” as used herein, represents a monovalent, monocyclic or fused ring bicyclic or multicyclic system having at least one heteroatom as a ring atom. For example, a heterocyclyl group may have, e.g., one to fifteen carbon ring atoms (e.g., a C1-C2, C1-C3, C1-C4, C1-C5, C1-C6, C1-C7, C1-C8, C1-C9, C1-C10, C1-C11, C1-C12, C1-C13, C1-C14, or C1-C15 heterocyclyl) and one or more (e.g., one, two, three, four, or five) ring heteroatoms independently selected from the group consisting of nitrogen, oxygen, and sulfur. Heterocyclyl groups may or may not include a ring that is aromatic. In preferred embodiments of the invention, a heterocyclyl group is a 3- to 8-membered ring, a 3- to 6-membered ring, a 4- to 6-membered ring, a 5-membered ring, or a 6-membered ring. Exemplary 5-membered heterocyclyl groups may have zero to two double bonds, and exemplary 6-membered heterocyclyl groups may have zero to three double bonds. Heterocyclyl groups may be substituted or unsubstituted. Exemplary substituents include alkyl, alkoxy, alkylthio, amido, amino, aryl, carbonate, carboxyl, cyano, epoxy, halo, heterocyclyl, hydroxyl, and thiol.

The term “individual” or “subject” is an animal, such as a mammal, bird, amphibian, or reptile. Mammals, as used herein, include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human.

The term “monomeric unit,” as used herein, refers to the radical residue formed from polymerization of the specified group.

The term “oxo,” as used herein, refers to ═O.

The term “pharmaceutical composition,” as used herein, refers to a preparation which is in such form as to permit the biological activity of an active ingredient contained therein to be effective, and which contains no additional components which are unacceptably toxic to a subject to which the formulation would be administered.

The term “pharmaceutically acceptable carrier,” as used herein, refers to an ingredient in a pharmaceutical composition, other than an active ingredient, which is nontoxic to a subject. A pharmaceutically acceptable carrier includes, but is not limited to, a buffer, excipient, stabilizer, or preservative.

The term “therapeutically effective amount,” as used herein, e.g., of a pharmaceutical composition, refers to an amount effective, at dosages and for periods of time necessary, to achieve the desired therapeutic or prophylactic result. A therapeutically effective amount of an agent for example eliminates, decreases, delays, minimizes, reduces, or prevents adverse effects of a disease.

The term “thiol,” as used herein, refers to —SH.

### BRIEF DESCRIPTION OF THE DRAWINGS

The following detailed description of the embodiments of the invention will be better understood when read in conjunction with the appended drawings. For the purpose of illustrating the invention, there are shown in the drawing embodiments, which are presently exemplified. It should be understood, however, that the invention is not limited to the precise arrangement and instrumentalities of the embodiments shown in the drawings.

FIG. 1 shows end-group modified cationic bottlebrush delivery vehicles complexed with pDNA forming complexes (e.g., in water or phosphate buffered saline (PBS)) that are used to transfect HEK-293 cells.

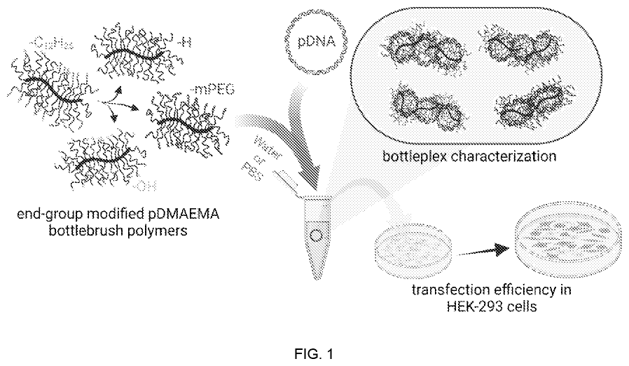

FIG. 2A, FIG. 2B, FIG. 2C, and FIG. 2D show stacked 1H nuclear magnetic resonance (NMR) spectra of end-group modified pDMAEMA bottlebrushes. FIG. 2A shows an NMR spectrum of BB-pDMAEMA-20-36-C₁₂H₂₅, FIG. 2B shows an NMR spectrum of BB-pDMAEMA-20-36-H, FIG. 2C shows an NMR spectrum of BB-pDMAEMA-20-36-OH, and FIG. 2D shows an NMR spectrum of BB-pDMAEMA-20-36-mPEG.

FIG. 3A shows light scattering and FIG. 3B shows differential refractive index SEC-MALS traces of bottlebrush polymers with DMF with 0.05M LiBr.

FIG. 4A, FIG. 4B, FIG. 4C, and FIG. 4D show BB-pDMAEMA-X stock solutions and complexes with pDNA at N/P of 7.5 in water. FIG. 4A shows the polymer only trace of BB-pDMAEMA-C₁₂H₂₅, FIG. 4B shows the polymer only trace of -pDMAEMA-H, FIG. 4C shows the polymer only trace of BB-pDMAEMA-OH, and FIG. 4D shows the polymer only trace of BB-pDMAEMA-mPEG.

FIG. 5A, FIG. 5B, FIG. 5C, and FIG. 5D show BB-pDMAEMA-X stock solutions and complexes with pDNA at N/P of 7.5 in PBS. FIG. 5A shows the polymer only trace of BB-pDMAEMA-C₁₂H₂₅, FIG. 5B shows the polymer only trace of BB-pDMAEMA-H, FIG. 5C shows the polymer only trace of BB-pDMAEMA-OH, and FIG. 5D shows the polymer only trace of BB-pDMAEMA-mPEG.

FIG. 6A, FIG. 6B, FIG. 6C, and FIG. 6D show aggregation studies as a function of time with the addition of OptiMEM. FIG. 6A shows an overlay of complexes formulated in water. FIG. 6B shows aggregation as function of time after the addition of OptiMEM to complexes in water. FIG. 6C shows an overlay of complexes formulated in PBS with respect to the size of pDNA alone in PBS. FIG. 6D shows aggregation of complexes formulated in PBS as a function of time upon the addition of OptiMEM.

FIG. 7A and FIG. 7B show dye exclusion binding assay of end-group modified bottlebrush polymers with pDNA formulated in water (FIG. 7A) and PBS (FIG. 7B) at N/P ratios of 5, 7.5, and 10.

FIG. 8A shows transfection results in water (solid bars) and PBS (hashed bars) as a function of percent green fluorescent protein (GFP) positive cells with all end-group modified BB-pDMAEMA-X delivery vehicles at N/P ratios of 5, 7.5, and 10 compared to controls of untreated cells, lipofectamine, pDNA treated cells, and JetPEI. BB-pDMAEMA-C₁₂H₂₅, BB-pDMAEMA-H, BB-pDMAEMA-OH, and BB-pDMAEMA-mPEG. FIG. 8B shows normalized cell viability of transfected samples acquired from CCK-8 assay.

FIG. 9A shows transfection results in water (solid bars) and PBS (hashed bars) as a function of % mCherry positive cells with all end-group modified BB-pDMAEMA-X delivery vehicles at N/P ratios of 5, 7.5, and 10 compared to controls of untreated cells, pDNA treated cells, and JetPEI. BB-pDMAEMA-X delivery vehicles are BB-pDMAEMA-C₁₂H₂₅, BB-pDMAEMA-H, BB-pDMAEMA-OH, and BB-pDMAEMA-mPEG. FIG. 9B shows normalized cell viability of transfected samples acquired from CCK-8 assay.

FIG. 10A, FIG. 10B, FIG. 10C, and FIG. 10D show aggregation studies of ribonucleoprotein (RNP) polymer complexes as a function of time with the addition of OptiMEM. FIG. 10A shows an overlay of complexes formulated in water. FIG. 10B shows aggregation as function of time after the addition of OptiMEM to complexes in water. FIG. 10C shows an overlay of complexes formulated in PBS with respect to the size of RNP alone in PBS. FIG. 10D shows aggregation of complexes formulated in PBS as a function of time upon the addition of OptiMEM.

FIG. 11A and FIG. 11B show dye exclusion binding assay of end-group modified bottlebrush polymers with RNP formulated in water (FIG. 11A) and PBS (FIG. 11B) at N/P ratios of 5, 7.5, and 10.

### DETAILED DESCRIPTION OF THE INVENTION

Polymeric delivery vehicles, e.g., cationic polymeric delivery vehicles, are advantageous platforms for nucleic acid delivery because of their scalability, biocompatibility, and chemical versatility. The invention allows for precisely tuned chemical functionality with various macromolecular architectures to increase the efficacy of nonviral-based gene delivery. Disclosed herein are polymeric delivery vehicles for the delivery of biological agents. The polymeric delivery vehicles may condense biological agents, such as CRISPR payloads (for example, mRNA, pDNA or ribonucleoproteins (RNP)), which can vary widely in their lengths, topologies, physical characteristics and biological mechanisms, into discrete nanosized polyelectrolyte complexes. Upon administration, the complexes may navigate both extracellular barriers such as serum DNAases (or RNAases) and reticuloendothelial system clearance, as well as intracellular barriers such as endosomal interrogation and lysosomal degradation. The biological agent may then be released within the spatiotemporal window that is optimal for payload translocation to the nucleus, where the biological agent can undergo further processing and realization of targeted edits. In addition to meeting high standards for safety, efficiency and cost-effectiveness, synthetic vectors may minimize immune activation and cellular toxicity.

In general, the present disclosure is directed to polymers that may be associated with at least one biological agent payload such as pDNA, RNP, and the like. The complexes may be internalized by a cell via various endocytic routes, the biological agent may be released inside the cell, and it subsequently may enter the cell nucleus to alter gene expression. The polymers disclosed herein thus provide a polymeric scaffold that provides a well-defined host configured to bind with biological macromolecular agents and facilitate intracellular delivery thereof. The polymers have physiochemical properties such as, for example, composition, molecular weight, Z-potential, pKa, polyplex diameter, nucleic acid condensation, and combinations thereof selected for efficient nucleic acid payload delivery using, for example, a CRISPR/Cas9 delivery process. The polymers and related complexes may also have good gene editing efficiency, cellular internalization, cytotoxicity, and combinations thereof.

### Polymers

The polymers of the present invention may include a backbone having a first end, a second end, and at least one side-chain.

The present invention is related to a polymer of formula (I):

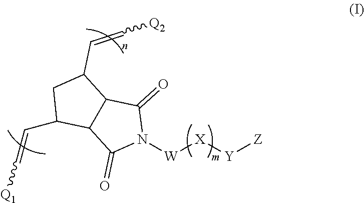

wherein, W is alkyl alkanoate; X is a monomeric unit of aminoalkyl acrylate; Y is a bond or S; Z is hydrogen, alkyl,

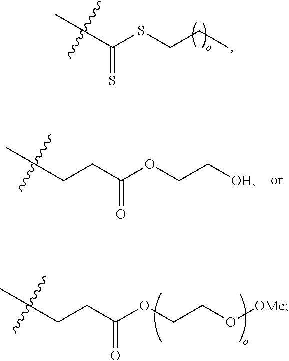

Q₁ is hydrogen or an end group; Q₂ is hydrogen or an end group; n is from 1 to 1000; m is from 1 to 1000; and o is from 1 to 100; or an ion or salt thereof. In some embodiments, Q₁ or Q₂ is an end group, such as alkyl, aryl, or heterocyclyl, e.g., phenyl. In some embodiments, Q₂ is hydrogen. In some embodiments, Q₁ is phenyl, and Q₂ is hydrogen.

In some embodiments, the compound of formula (I) is selected from:

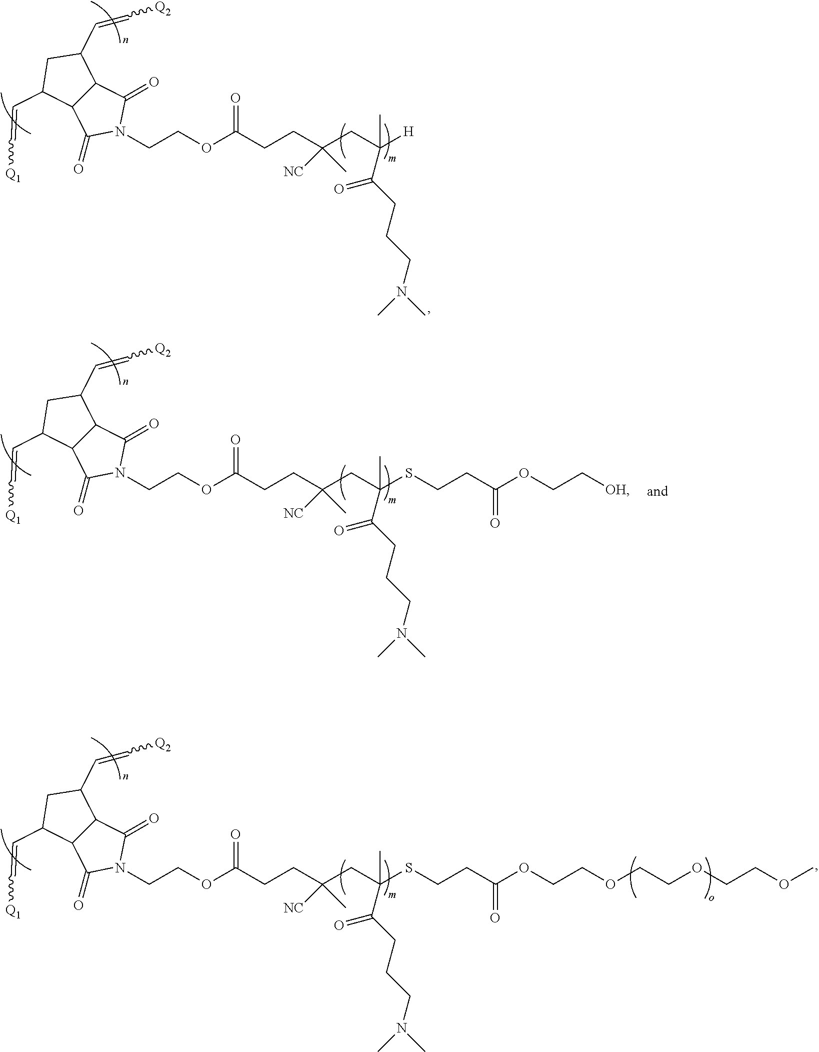

or an ion or salt thereof. In embodiments, Q₁ and Q₂ may be both H. In other embodiments, Q₁ may be phenyl, and Q₂ is H.

The present invention is further related to a compound of formula (II):

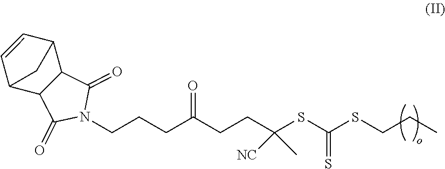

wherein o is from 1 to 20.

The present invention is further related to a polymer of formula (III):

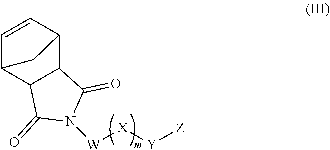

wherein W is alkyl alkanoate; X is a monomeric unit of aminoalkyl acrylate; Y is a bond or S;
Z is hydrogen, alkyl,

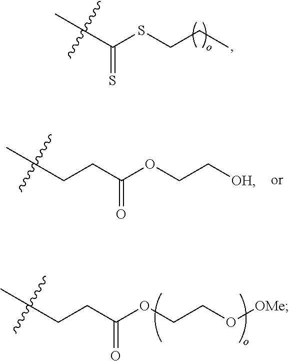

m is from 1 to 1000; and o is from 1 to 100; or an ion or salt thereof.

In formulas (I) and (III), m may be from about 1 to about 1000, e.g., from 1 to 2, 1 to 5, 1 to 10, 1 to 20, 1 to 25, 1 to 30, 1 to 40, 1 to 50, 1 to 60, 1 to 70, 1 to 75, 1 to 80, 1 to 90, 1 to 100, 1 to 125, 1 to 150, 1 to 175, 1 to 200, 1 to 250, 1 to 300, 1 to 400, 1 to 500, 1 to 600, 1 to 700, 1 to 800, 1 to 900, 2 to 5, 2 to 10, 2 to 25, 2 to 50, 5 to 10, 5 to 15, 5 to 20, 5 to 25, 5 to 30, 5 to 35, 5 to 40, 5 to 45, 5 to 50, 5 to 55, 5 to 60, 5 to 70, 5 to 75, 5 to 80, 5 to 90, 5 to 100, 10 to 20, 10 to 25, 10 to 30, 10 to 40, 10 to 50, 10 to 60, 10 to 70, 10 to 80, 10 to 90, 10 to 100, 15 to 20, 15 to 25, 15 to 30, 15 to 45, 16 to 24, 17 to 23, 18 to 22, 19 to 21, 20 to 30, 20 to 40, 20 to 50, 20 to 60, 20 to 70, 20 to 75, 20 to 80, 20 to 90, 20 to 100, 25 to 30, 25 to 35, 25 to 40, 25 to 50, 25 to 75, 25 to 100, 25 to 125, 26 to 34, 27 to 33, 28 to 32, 29 to 31, 30 to 35, 30 to 40, 30 to 45, 30 to 50, 30 to 60, 30 to 70, 30 to 80, 30 to 90, 30 to 100, 35 to 65, 40 to 50, 40 to 60, 40 to 70, 40 to 80, 40 to 90, 40 to 100, 50 to 60, 50 to 70, 50 to 80, 50 to 90, 50 to 100, 50 to 125, 50 to 150, 50 to 175, 50 to 200, 50 to 250, 50 to 300, 50 to 350, 50 to 400, 50 to 450, 50 to 500, 50 to 550, 50 to 600, 50 to 700, 50 to 800, 50 to 900, 50 to 1000, 60 to 100, 60 to 140, 70 to 100, 70 to 130, 80 to 100, 80 to 120, 90 to 110, 100 to 125, 100 to 150, 100 to 200, 100 to 300, 100 to 500, 100 to 750, 100 to 1000, 150 to 200, 150 to 250, 200 to 300, 200 to 500, 200 to 700, 200 to 1000, 250 to 500, 250 to 750, 250 to 1000, 300 to 500, 300 to 700, 400 to 500, 400 to 600, 500 to 600, 500 to 750, 500 to 800, 500 to 1000, 750 to 1000, 800 to 900, 800 to 1000, or 900 to 1000.

In formulas (I) and (III), n may be from about 1 to about 1000, e.g., from 1 to 2, 1 to 5, 1 to 10, 1 to 20, 1 to 25, 1 to 30, 1 to 40, 1 to 50, 1 to 60, 1 to 70, 1 to 75, 1 to 80, 1 to 90, 1 to 100, 1 to 125, 1 to 150, 1 to 175, 1 to 200, 1 to 250, 1 to 300, 1 to 400, 1 to 500, 1 to 600, 1 to 700, 1 to 800, 1 to 900, 2 to 5, 2 to 10, 2 to 25, 2 to 50, 5 to 10, 5 to 15, 5 to 20, 5 to 25, 5 to 30, 5 to 35, 5 to 40, 5 to 45, 5 to 50, 5 to 55, 5 to 60, 5 to 70, 5 to 75, 5 to 80, 5 to 90, 5 to 100, 10 to 20, 10 to 25, 10 to 30, 10 to 40, 10 to 50, 10 to 60, 10 to 70, 10 to 80, 10 to 90, 10 to 100, 15 to 20, 15 to 25, 15 to 30, 15 to 45, 16 to 24, 17 to 23, 18 to 22, 19 to 21, 20 to 30, 20 to 40, 20 to 50, 20 to 60, 20 to 70, 20 to 75, 20 to 80, 20 to 90, 20 to 100, 25 to 30, 25 to 35, 25 to 40, 25 to 50, 25 to 75, 25 to 100, 25 to 125, 26 to 34, 27 to 33, 28 to 32, 29 to 31, 30 to 35, 30 to 40, 30 to 45, 30 to 50, 30 to 60, 30 to 70, 30 to 80, 30 to 90, 30 to 100, 35 to 65, 40 to 50, 40 to 60, 40 to 70, 40 to 80, 40 to 90, 40 to 100, 50 to 60, 50 to 70, 50 to 80, 50 to 90, 50 to 100, 50 to 125, 50 to 150, 50 to 175, 50 to 200, 50 to 250, 50 to 300, 50 to 350, 50 to 400, 50 to 450, 50 to 500, 50 to 550, 50 to 600, 50 to 700, 50 to 800, 50 to 900, 50 to 1000, 60 to 100, 60 to 140, 70 to 100, 70 to 130, 80 to 100, 80 to 120, 90 to 110, 100 to 125, 100 to 150, 100 to 200, 100 to 300, 100 to 500, 100 to 750, 100 to 1000, 150 to 200, 150 to 250, 200 to 300, 200 to 500, 200 to 700, 200 to 1000, 250 to 500, 250 to 750, 250 to 1000, 300 to 500, 300 to 700, 400 to 500, 400 to 600, 500 to 600, 500 to 750, 500 to 800, 500 to 1000, 750 to 1000, 800 to 900, 800 to 1000, or 900 to 1000.

In formulas (I) and (III), o may be from about 1 to about 100, e.g., from 1 to 2, 1 to 5, 1 to 10, 1 to 20, 1 to 25, 1 to 30, 1 to 40, 1 to 50, 1 to 60, 1 to 70, 1 to 75, 1 to 80, 1 to 90, 2 to 5, 2 to 10, 2 to 25, 2 to 50, 5 to 10, 5 to 15, 5 to 20, 5 to 25, 5 to 30, 5 to 35, 5 to 40, 5 to 45, 5 to 50, 5 to 55, 5 to 60, 5 to 70, 5 to 75, 5 to 80, 5 to 90, 5 to 100, 10 to 20, 10 to 25, 10 to 30, 10 to 40, 10 to 50, 10 to 60, 10 to 70, 10 to 80, 10 to 90, 10 to 100, 15 to 20, 15 to 25, 15 to 30, 15 to 45, 16 to 24, 17 to 23, 18 to 22, 19 to 21, 20 to 30, 20 to 40, 20 to 50, 20 to 60, 20 to 70, 20 to 75, 20 to 80, 20 to 90, 20 to 100, 25 to 30, 25 to 35, 25 to 40, 25 to 50, 25 to 75, 25 to 100, 25 to 125, 26 to 34, 27 to 33, 28 to 32, 29 to 31, 30 to 35, 30 to 40, 30 to 45, 30 to 50, 30 to 60, 30 to 70, 30 to 80, 30 to 90, 30 to 100, 35 to 65, 40 to 50, 40 to 60, 40 to 70, 40 to 80, 40 to 90, 40 to 100, 50 to 60, 50 to 70, 50 to 80, 50 to 90, 50 to 100, 60 to 100, 70 to 100, 80 to 100, or 90 to 100, e.g., is about 10.

In formula (II), o may be from about 1 to about 20, e.g., from 1 to 2, 1 to 5, 1 to 10, 1 to 15, 2 to 5, 2 to 10, 2 to 15, 5 to 10, 5 to 15, 5 to 20, 6 to 14, 7 to 13, 8 to 12, 9 to 11, 10 to 15, 10 to 20, 15 to 20, 16 to 20, 17 to 20, 18 to 20, or 19 to 20, e.g., is about 10.

In some embodiments, the aminoalkyl acrylate is 2-(dimethylamino)ethyl methacrylate (DMAEMA).

In some embodiments, side chains include a substantially equivalent number of repeating cationic units. In other words, each side-chain of a plurality of side-chains may have the same degree of polymerization (N_{sc}). In some examples, N_{sc} may be from 2 to 1000, for example between about 20 and about 250, or between about 30 and about 70.

Alternatively, in some embodiments, N_{sc} may vary among individual side-chains of a plurality of side-chains, changing the architecture of the polymer structure. For example, the backbone may define a first end and a second end, and the polymer may define a first side-chain covalently bonded to a first portion of the backbone adjacent to the first end of the backbone and a second side-chain covalently bonded to a second portion of the backbone adjacent to the second end of the backbone. In some embodiments, the first side-chain may include a greater number of repeating cationic units (i.e., a larger N_{sc}) than the second side-chain. In this way, the polymer may define a “cone” shape, which may be desirable for delivery of certain biological agents. In another embodiment, the backbone may define a first end and a second end, and the polymer may define a first side-chain covalently bonded to a first portion of the backbone adjacent to the first end of the backbone, a second side-chain covalently bonded to a second portion of the backbone adjacent to the second end of the backbone, and a third side chain covalently bonded to a third portion of the backbone between the first end and the second end. The third side-chain may include a greater number of repeating cationic units (i.e., a larger N_{sc}) than the first side-chain and the second side-chain. In this way, the polymer may define a “football” shape, which may be desirable for delivery of certain biological agents. Although these first, second, and third side-chains are described for illustrative purposes, more side-chains may also be provided with varying lengths in order to achieve similar shapes on a larger scale, such as a plurality of side chains with increasing lengths from one end of the backbone to the other end of the backbone, or a plurality of side chains with the shortest side-chains at the ends of the backbone with increasing lengths of side-chains towards the middle of the backbone such as the longed side-chains are located near the middle of the backbone. In other examples, other variations on the lengths of the side-chains along the length of the backbone may be used to produce different shapes of a polymer described herein. For example, more “cylindrical” shaped polymers may include a relatively longer backbone having side-chains with consistent lengths, which may be desirable for delivery of certain biological agents.

In some examples, the polymers may be characterized by number-average molecular weight, Mn, pKa, or zeta potential. For example, a polymer may have an Mn from about 10 kilodaltons (kDa) to about 1000 kDa, such as from about 100 kDa to about 400 kDa. In some examples, a polymer may have a pKa, which may be defined as the negative base ten logarithm of the acid dissociation constant Ka, of from about 6.0 to about 9.0, such as from about 6.9 to about 7.0. In some examples, a polymer may have a zeta potential, Z-potential, of about 10 millivolts (mV) to about 40 mV.

### Complexes

Polymeric delivery vehicles are a versatile platform for the delivery of biological agents, and offer numerous advantages over viral vectors by potentially enabling lower immunogenicity and production costs along with facile scalability.

The polymers described herein may bind with a biological agent. The biological agent may be bound to the polymer in a variety of methods. Without wishing to be bound by theory, cationic polymers may readily complex negatively charged biological agents through an entropically driven displacement of counterions to form interpolyelectrolyte complexes. In some embodiments, the biological agent is bound noncovalently to the polymer, e.g., electrostatically to the polymer. In some embodiments, the polymer may be complexed with the biological agent. In some embodiments, the polymer may be condensed with the biological agent. Without wishing to be bound to theory, the bottlebrush polymers described herein, including those bound, complexed, or condensed to negatively charged biological agents, may be able to evade the immune system by mimicking bacteria-like, less-foreign morphologies, thereby showing great promise as a multiplexable system.

The present disclosure provides a complex including a polymer of formula (I) or (III), and a biological agent. In some embodiments, the biological agent is a negatively charged biological agent. The biological agent may include a therapeutic agent. The biological agent may include a nucleic acid, a peptide, a protein, or a small molecule.

The biological agent may include a small molecule. Small molecules are compounds with low molecular weight that are capable of modulating biochemical processes to diagnose, treat, or prevent diseases. In some examples, polymers may deliver small molecule therapeutics by covalent binding and/or noncovalent sequestration of the small molecule. The present polymeric delivery systems may be used to transport small-molecule therapeutics to cells.

Delivery of large biological payloads may be completed using polymers of the present disclosure. The biological agent may include a nucleic acid. Without wishing to be bound to theory, when negatively charged biological agents (e.g., nucleic acids such as RNA or other small molecule therapeutics) are covalently attached to the bottlebrush architecture, the circulation time of the cargo may be increased in vivo. The nucleic acid may be DNA, RNA, or chimeric. In some embodiments, the nucleic acid includes gRNA, mRNA (e.g., that encodes for proteins (fluorescence or therapeutic), tmRNA, tRNA, rRNA, siRNA, shRNA, PNA, ssRNA, dsRNA, pDNA (e.g., that encodes for proteins (fluorescence or therapeutic), ssDNA, dsDNA, a DNA: RNA hybrid molecule, DNA editing templates, miRNA, an artificial chromosome, oligo nucleotide, a nucleic acid encoding a nuclease, cDNA, a PCR product, a restriction fragment, a ribozyme, an antisense construct, or a combination thereof. Nucleic acids may include modification to the sugar, backbone, and/or base and may include synthetic or non-canonical bases.

A gRNA includes an engineered, non-naturally occurring Clustered Regularly Interspaced Short Palindromic Repeats (CRISPR) gRNA that hybridizes with a target nucleic acid sequence of interest.

In various embodiments, DNA editing templates include an exogenous strand of DNA that bears homology arms to a section of genomic DNA that has been cut by a nuclease (for example, Cas9, TALEN or zinc finger) along with an intervening sequence between these homology arms that differs with the natural segment of genomic DNA that has been cut. This intervening segment serves as the template for repair of the cut genomic DNA and, in so doing, the cell corrects its own DNA to match that of the DNA template. The DNA template may be included in a single DNA expression vector that also encodes the nuclease.

siRNAs refer to a double-stranded interfering RNA. In addition to siRNA molecules, other interfering RNA molecules and RNA-like molecules may be used. Examples of other interfering RNA molecules that may to inhibit target biomolecules include, but are not limited to, short hairpin RNAs (shRNAs), single-stranded siRNAs, microRNAs (miRNAs), piwiRNA, Dicer-substrate 27-mer duplexes, and variants thereof containing one or more chemically modified nucleotides, one or more non-nucleotides, one or more deoxyribonucleotides, and/or one or more non-phosphodiester linkages. Typically, all RNA or RNA-like molecules that may interact with transcripts RISC complexes and participate in RISC-related changes in gene expression may be referred to as interfering RNAs or “interfering RNA molecules.

Unlike siRNA and oligonucleotides, pDNA payloads may present unique challenges as the long semiflexible structure may impose additional constraints on their polymeric binding partners during polymer-pDNA assembly and compaction. Moreover, unlike other nucleic acid payloads, pDNA may require delivery to the nucleus to accomplish its therapeutic function. The disclosure provides polymer systems which have unique architectural and morphological features to overcome delivery challenges of large biological payloads including pDNA.

Suitable interfering RNAs may readily be produced based on the well-known nucleotide sequences of target biomolecules. In various embodiments interfering RNAs that inhibit target biomolecules may include partially purified RNA, substantially pure RNA, synthetic RNA, recombinant produced RNA, as well as altered RNA that differs from naturally occurring RNA by the addition, deletion, substitution, and/or alteration of one or more nucleotides. Such alterations may include, for example, addition of non-nucleotide material, such as to the end(s) of the interfering RNAs or to one or more internal nucleotides of the interfering RNAs, including modifications that make the interfering RNAs resistant to nuclease digestion. Such alterations result in sequences that are generally at least about 80%, or more, or even 100% identical to the sequence of the target biomolecule. When the gene to be down regulated is in a family of highly conserved genes, the sequence of the duplex region may be chosen with the aid of sequence comparison to target only the desired gene. On the other hand, if there is sufficient identity among a family of homologous genes within an organism, a duplex region may be designed that would down regulate a plurality of genes simultaneously.

The N/P ratio of a complex is the ratio of positively chargeable polymer amine (N=nitrogen) groups to negatively-charged nucleic acid phosphate (P) groups. The N/P character of a polymer/nucleic acid complex may influence complex properties, such as its net surface charge, size, and stability of the complex. The N/P ratio of the present complexes may be from 0 to 10, e.g., from 0 to 1, 0 to 2, 0 to 3, 0 to 4, 0 to 5, 0 to 6, 0 to 7, 0 to 8, 0 to 9, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, 9 to 10, or about 0.5, about 1, about 2, about 3, about 4, about 5, about 6, about 7, about 8, about 9, or about 10.

Alternatively, or in addition, the biological agent may include a peptide. In various embodiments, peptide fragments include two or more amino acids covalently linked by at least one amide bond. For example, in some embodiments the peptide fragments may include pDNA encoded fluorescence or therapeutic peptides.

Alternatively, or in addition, the biological agent may include a protein. In some embodiments, the protein may include an antibody. The antibody may be a monoclonal antibody.

In some embodiments, the protein includes a ribonucleoprotein. The ribonucleoprotein may include a ribosome, telomerase, ribonuclease P (RNase P), a heterogeneous ribonucleoprotein particle (hnRNP), or a small nuclear ribonucleoprotein particle (snRNP). sgRNA may be chemically modified to improve stability and prevent intracellular degradation.

In some embodiments, the protein may include a nuclease. The nuclease may include a zinc finger nuclease (ZFNs), a transcription-activator like effector nucleases (TALEN), or a Cas protein. The Cas protein may be a Cas2, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas12, Cas13, Cas14, CfpI, CasI, CasIB, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, modified versions thereof, or combinations thereof. In some embodiments, the Cas protein is Cas9.

In some embodiments, the biological agent includes a nucleic acid and a nuclease. The negatively charged biological agent may be gRNA and a Cas protein, as in the CRISPR-Cas system. The CRISPR-Cas system is useful for precise editing of genomic nucleic acids (e.g., for creating null mutations). For example, a composition containing only the guide RNA can be administered to an animal or cells transgenic for the Cas9 enzyme. Similar strategies may be used (e.g., zinc finger, transcription activator-like effectors (TALEs) or homing meganucleases).

The CRISPR-Cas system is known in the art for deleting, modifying genome sequences or incorporating transgenes. Transgene refers to any nucleotide sequence, particularly a DNA sequence, that is integrated into one or more chromosomes of a host cell by human intervention, such as by the methods of the present invention. For example, a transgene can be an RNA coding region or a gene of interest, or a nucleotide sequence, preferably a DNA sequence, that is used to mark the chromosome where it has integrated or may indicate a position where nucleic acid editing, such as by the CRISPR-CAS system, may occur. In this situation, the transgene does not have to include a gene that encodes a protein that may be expressed. CRISPR-Cas genome editing has rapidly emerged as a multi-faceted technology to enable gene insertion, deletion, activation, suppression, and even single base editing of target genes within the nucleus of any cell. This highly efficient and facile technique has broad utility from white biotechnology and agriculture to biomedical research, pharmaceutics, and regenerative medicine.

Currently, the CRISPR/Cas9 system can be delivered in vitro, ex vivo, and in vivo in three different payload forms: i) pDNA that encodes Cas9 protein and/or sgRNA, ii) mRNA that encodes for Cas9 nuclease and a separate sgRNA, or iii) a ribonucleoprotein (RNP) that consists of recombinant Cas9 protein precomplexed directly with a sgRNA.

CRISPR-Cas9 pDNA needs to enter the cellular nucleus to express, and consistent expression produces an overabundance of Cas9 protein, which can lead to increased off-target editing and mutagenesis. Researchers have utilized the CRISPR/Cas9 system in mRNA form to circumvent the barrier of nuclear entry, which has been reported with polymer-based nanoparticles. However, sgRNA often needs to be delivered separately, presenting challenges in trafficking kinetics of different payloads.

Direct delivery of CRISPR/Cas9 ribonucleoprotein (RNP) has several benefits, including precision in endonuclease dosing and potential to avoid uncontrolled integration of the transgene into the cellular genome.

### Formulations

In another aspect, the present disclosure is directed to compositions including the polymer complexes described above which have been dispersed in a solution. In some embodiments, the composition includes a plurality of polymers described herein which have been dispersed in a solution, e.g., a mixture of 2, 3, 4, 5, or more. In some embodiments, the complexes may be added to a liquid carrier and stored in liquid form until needed, or alternatively may be dried and introduced into and dispersed in the liquid carrier prior to use, e.g., administration to a subject.

In some embodiments the liquid carrier is a pharmaceutically acceptable carrier. Some non-limiting examples of materials which can serve as pharmaceutically-acceptable carriers include: pyrogen-free water; isotonic saline; Ringer's solution; ethyl alcohol; pH buffered solutions. The formulation may include solvents, dispersing medium (containing, e.g., water, cell culture medium, buffers (e.g., phosphate buffered saline), polyol (for example, glycerol, propylene glycol, or liquid polyethylene glycol), wetting agents, emulsifying agents, coloring agents, release agents, coating agents, sweetening agents, flavoring agents, perfuming agents, pH buffering agents, gelling or viscosity enhancing additives, preservatives, colors, preservatives, or antioxidants.

The compounds of formula (I) and (III) may be ionized in dry or liquid formulation. For example, amine or basic nitrogen groups may be protonated. The compounds of formula (I) and (III) may be in salt form with one or more anions, e.g., acetate, ascorbate, benzoate, bicarbonate, bisulfate, carbonate, cholate, citrate, dihydrogen citrate, glycocholate, halide (such as F, Cl, Br, or I), hydrogen citrate, hydroxide, mandelate, methanesulfonate, nitrate, oxalate, p-toluenesulfonate, persulfate, phosphate, lactate, succinate, sulfate, sulfite, tartrate, taurocholate, or trifluoroacetate.

### Methods of Use

The present disclosure provides methods of contacting a cell with the herein-described complexes, wherein the biological agent is delivered into the cell.

For example, after a composition including the polymer and bound biological agent are contacted with a cell, the complexes internalize into the cell, and the biological agent disassociate partially or completely from the polymeric carriers.

The formulations described herein can be delivered to a cell or an organism via any administration mode known to a skilled practitioner. For example, the formulations described herein can be delivered via administration routes such as, but not limited to, in vitro, oral, intravenous, intramuscular, intraperitoneal, intradermal, and subcutaneous. In some embodiments, the compositions described herein are in a form that is suitable for injection. In other embodiments, the formulations described herein are formulated for oral administration. In various embodiments, for in vivo administration a delivery device can be used to facilitate the administration of any composition described herein to a subject, e.g., a syringe, a dry powder injector, a nasal spray, a nebulizer, or an implant such as a microchip, e.g., for sustained release or controlled release of any formulation described herein.

In various embodiments, the compositions may be administered to a cell in vitro by removing a cell from a subject, culturing the cells, applying to the cells a composition including polymer vehicles and bonded biological agent to deliver a therapeutic amount of the biological agent into at least a portion of the cells, and optionally re-introducing the cell to the subject.

In another embodiment, a tissue cell therapy technique may be used in which a tissue sample is removed from a subject, a composition including a polymer and a bonded biological agent is applied to the tissue to deliver a therapeutic amount of the biological agent to modify a selected cell or region of the tissue, and the modified tissue is transplanted into the subject.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1: Synthesis of pDMAEMA Bottlebrushes Having End-Group Modifications

A template 2-(dimethylamino)ethyl methacrylate bottlebrush (pDMAEMA) was synthesized using a norbornene-functionalized chain transfer agent (NB-CTA) to homopolymerize methacrylate-based monomers via reversible addition-fragmentation chain-transfer (RAFT) polymerization. The synthesis afforded a BB-pDMAEMA-20-30-C₁₂H₂₅ with 20 pDMAEMA repeat units in the side chains, a backbone degree of polymerization of 30, and a dodecyl hydrocarbon end-group attached to the trithiocarbonate, as shown in Table 1.

TABLE 1

| Sample | Mₙ (kDa) | M_{w} (kDa) | Ð |
|---|---|---|---|
| BB-pDMAEMA-20-30-C₁₂H₂₅ | 114 | 130 | 1.14 |
| BB-pDMAEMA-20-30-H | 102 | 117 | 1.14 |
| BB-PDMAEMA-20-30-OH | 121 | 142 | 1.18 |
| BB-pDMAEMA-20-30-mPEG | 107 | 123 | 1.16 |

Post-polymerization end-group modification techniques were used to remove the trithiocarbonate end-group or install hydroxyethyl acrylate and methyl polyethylene glycol acrylate end-groups to increase the colloidal stability of the bottlebrushes in aqueous media, as shown in Scheme 1. End-group modifications to the template BB-pDMAEMA-20-36-C₁₂H₂₅ were achieved using photoinduced chain transfer removed the trithiocarbonate completely and aminolysis followed by thiol-Michael addition of hydroxyethyl acrylate or methyl polyethylene glycol acrylate.

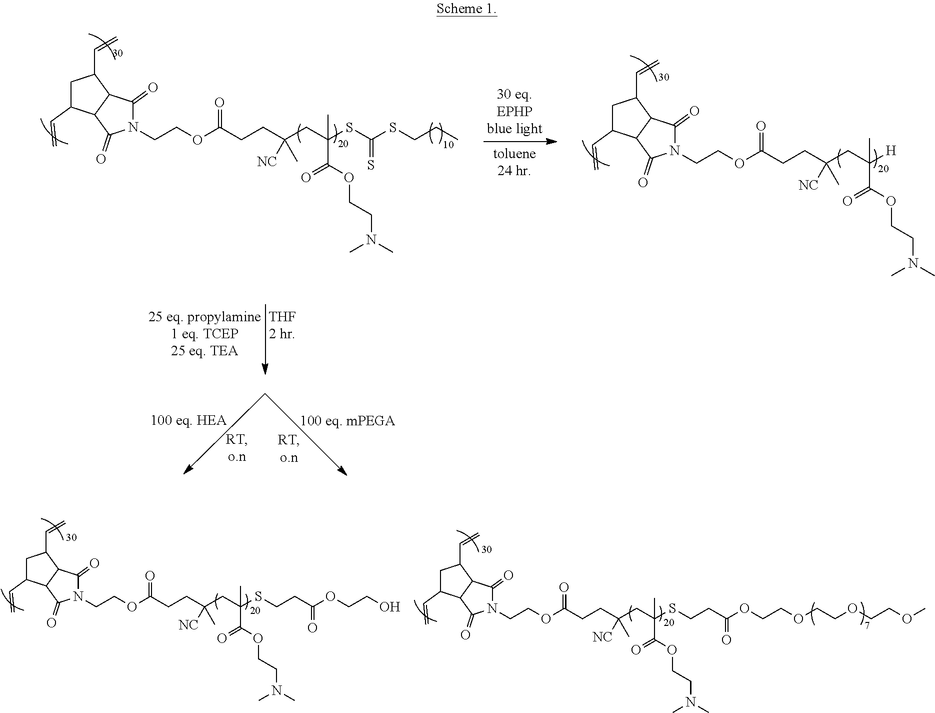

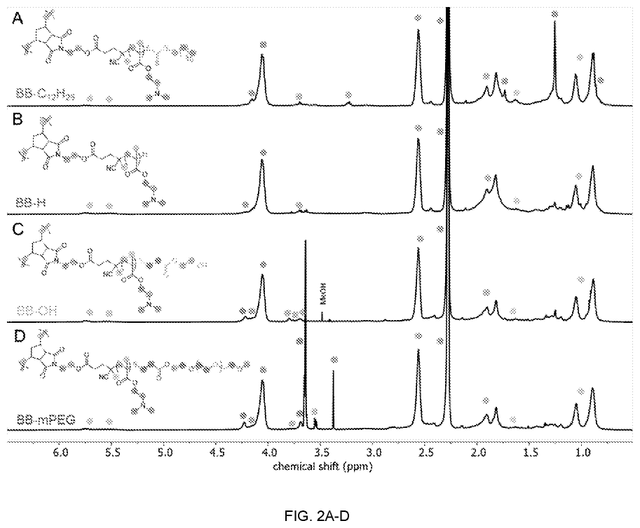

To completely remove the trithiocarbonate end-groups, the reaction was run in toluene only with equivalents of ethylpiperidine hypophosphite (EPHP) per end-group for only 24 hours. Running the reaction for longer, in the presence of methanol, and with more EPHP lead to the cleavage of side chains from the backbone decreasing the molecular weight significantly. The optimized conditions resulted in only cleavage of the trithiocarbonate end-groups were visualized by proton NMR in FIG. 2A and FIG. 2D by the disappearance of shifts at 1.25, 1.76, and 3.22 ppm.

Aminolysis and thiol-Michael addition was optimized by using triethylamine (TEA) to initially catalyze the aminolysis reaction and cleaving the trithiocarbonate to a thiol with 25 equivalents of propylamine over 2 hours. The reactions were then stirred overnight with 100 equivalents of either hydroxy ethyl acrylate (HEA) or polyethylene glycol ethyl ether acrylate (PEGA) to end-cap each side chain with the desired end-group. The disappearance of the dodecyl hydrocarbon shifts at 1.25, 1.76, and 3.22 ppm observed by proton NMR indicated the cleavage of the trithiocarbonate to a thiol. The quantitative functionalization of all end-groups with HEA and PEGA were calculated using the 4 proton shifts at 3.79-3.68 ppm and 3 protons of the methyl group on the PEGA at 3.37 ppm, respectively, as shown in FIGS. 2A-2D. Color change of all end-group modified samples from yellow to off white was observed as the trithiocarbonate was removed.

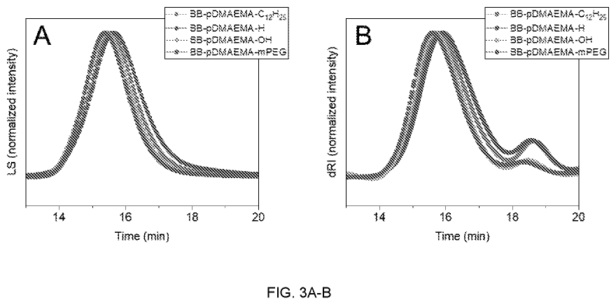

Each sample was characterized by size exclusion chromatography in dimethylformamide (DMF) with 0.05M LiBr, as shown in Table 1, before protonation with hydrochloric acid (HCl) and dialyzed in MilliQ water. The overlayed light scattering and differential refractive index traces of each end-group modified bottlebrush polymer are shown in FIG. 3A and FIG. 3B. Each of the traces overlay well with one another with respect to retention time indicating no side chain cleavage or coupling of bottlebrush polymers during end-group modification. Because each end-group modified bottlebrush was synthesized from the same template, the influence of end-group chemistry on plex formation, stability, and transfection efficiency can be compared directly.

### Example 2: Influence of End-Groups on Solution Behavior and Complex Formation

After synthetic characterization, each bottlebrush polymer transfection agent was protonated with HCl and dialyzed against MilliQ water before being dried on the lyophilizer. The small library of end-group modified pDMAEMA bottlebrush polymers was then complexed using electrostatic interactions with pDNA in water and PBS to transfect HEK-293 cells with pDNA encoding for green fluorescent protein (GFP). To understand the structure-function relationship of these end-group modified BB-pDMAEMA-X transfection agents when complexed with pDNA in solution, the complexes were characterized using dynamic light scatting before and after complexation with pDNA. It is well understood that polyplex formulations aggregate in solution with the addition of OptiMEM, a nutrient rich buffer added during the transfection protocol. Differences in the aggregation rate and size were observed between formulation media and end-group chemistry of the pDMAEMA bottlebrush.

### Dynamic Light Scattering

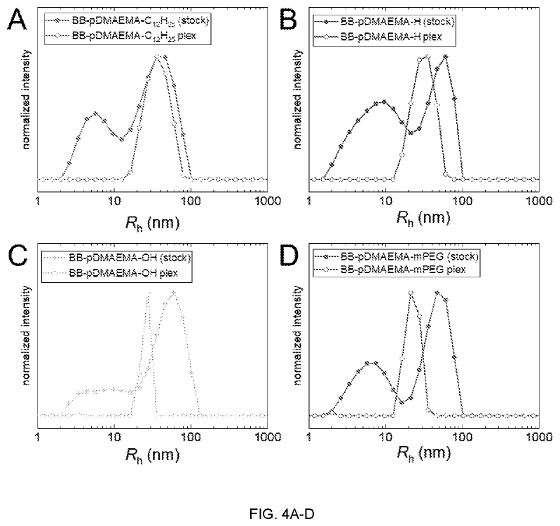

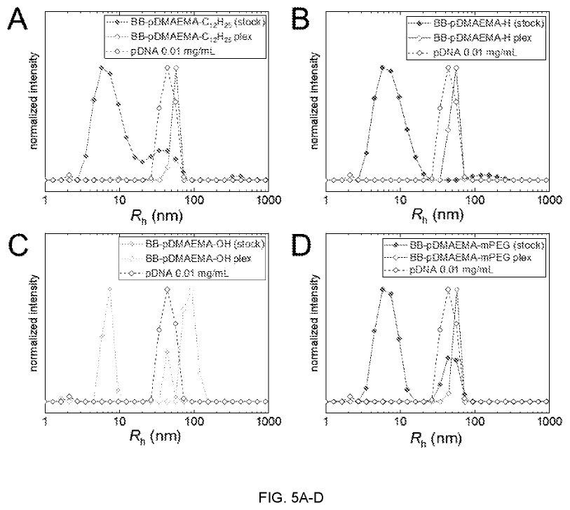

The hydrodynamic radius of each end-group modified bottlebrush was investigated in solution using dynamic light scattering to identify if the samples are aggregating or if they exist as monomodal populations in solution. As shown in FIG. 4A, FIG. 4B, FIG. 4C, FIG. 4D, as well as in FIG. 5A, FIG. 5B, FIG. 5C, and FIG. 5D, the polymer only trace (filled in circles or diamonds) was measured from the stock solution of approximately 2.6-3.0 mg/mL which was diluted to the correct N/P ratio before complex formation. The stock solutions were used due to low scattering intensity of the N/P=7.5 relevant dilutions in water and PBS on the Wyatt plate reader dynamic light scattering (DLS) system. The concentrations of the N/P=7.5 dilutions ranged from 0.08-0.09 mg/mL, and the scattering intensity of the bottlebrush polymers in solution was too low to accurately measure. A Brookhaven DLS setup allowed the varying of laser power to achieve the appropriate number of counts to properly baseline an autocorrelation function for the dilute solutions. When complexed with pDNA, the complexes scattered strongly at the desired N/P ratio due to their size and were measured without issue.

In water, the bottlebrush stock solutions all aggregated with the normalized intensity vs. hydrodynamic radius trace highlighting a population of single bottlebrush polymers with Rh of 5-10 nm and an aggregated population ranging from 36-61 nm. Upon complexation with pDNA, the complexes formed rapidly (within less than 5 minutes) to form polyelectrolyte complexes that exist as a monomodal population with sizes decreasing from 36 to 28 to 21 nm in Rh as the end-groups are changed from —C₁₂H₂₅ and —H to —OH to -mPEG, respectively. Each complex decreased in size compared to the aggregated population present in the stock solution. The BB-pDMAEMA-C₁₂H₂₅ and —H complexes are larger than the complexes formed from pDMAEMA bottlebrushes with —C₃H₇ end-groups. However, upon addition of HEA and PEGA the complex size incrementally decreased suggesting added colloidal stability afforded by the more hydrophilic end-groups.

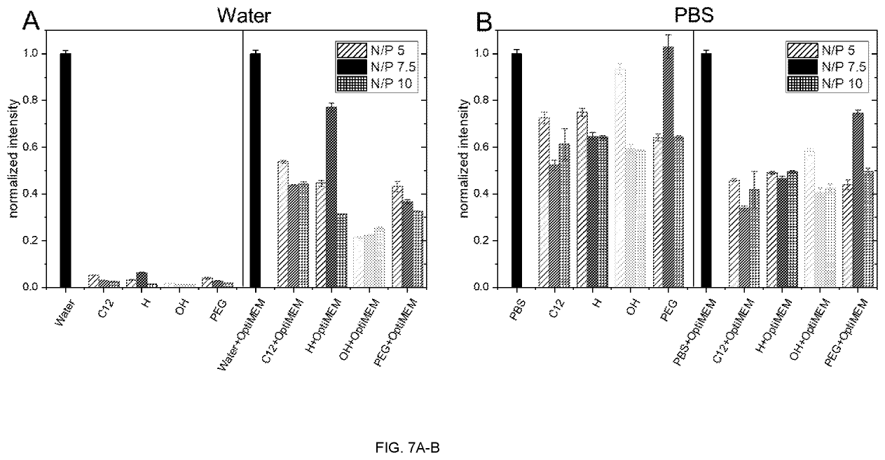

The end-group modified bottlebrush polymers formulated in PBS were measured to exist mainly as monomodal populations of free, non-aggregated, bottlebrushes in the stock solutions with Rh of 5.8 nm for all samples other than BB-pDMAEMA-OH with a larger Rh of 7.4 nm, as seen in FIG. 5A-5D. Without wishing to be bound to theory, buffered pH of PBS equal to 7.4 may lead to the bottlebrushes not being as positively charged in solution as they are in MilliQ water that has a pH of 6.9. The expected pKa for these systems is around 7.0. Therefore, as is seen in FIG. 7, the exclusion of dye due to pDNA binding in PBS is very low compared to in water. This lack of complete cationic nature could be contributing to the complexes sizes in PBS to be very similar to that of pDNA alone in PBS as seen in FIG. 5A, FIG. 5B, and FIG. 5D. The size of the complex formed with BB-pDMAEMA-OH in PBS at N/P of 7.5 is larger than the rest with an Rh of 93 nm compared to 57 nm (FIG. 5C and Table 2). To gain a better understanding of how these complexes react to the transfection conditions, the samples were diluted with OptiMEM and the aggregation overtime was measured.

TABLE 2

| | | | | Agg. Rate in | | | | | Agg. Rate in | |
|---|---|---|---|---|---|---|---|---|---|---|
| | Rₕ, | | | water + | | Rₕ, | | | PBS + | |
| | stock, | Rₕ, plex, | | OptiMEM | | stock, | Rₕ, plex, | | OptiMEM | |
| | water | water (nm)^{a} | | (nm/min)^{b} | | PBS | PBS (nm)^{a} | | (nm/min)^{b} | |
| Sample | (nm)^{a} | pDNA | RNP | pDNA | RNP | (nm)^{a} | pDNA | RNP | pDNA | RNP |
| BB- | 5.7, 36 | 36 | 98 | 13 | 24 | 5.8, 34 | 57 | 370 | 8.0 | 220 |
| pDMAEMA- | | | | | | | | | | |
| C₁₂H₂₅ | | | | | | | | | | |
| BB- | 9.6, 61 | 36 | 76 | 8.8 | 11 | 5.8 | 56 | 170 | 6.4 | 4.5 |
| pDMAEMA- | | | | | | | | | | |
| H | | | | | | | | | | |
| BB- | 9.6, 61 | 28 | 99 | 7.2 | 14 | 7.4, 43 | 93 | 170 | 9.2 | 8.4 |
| pDMAEMA- | | | | | | | | | | |
| OH | | | | | | | | | | |
| BB- | 7.4, 47 | 21 | 76 | 1.0 | 13 | 5.8, 43 | 57 | 7.1 | 2.6 | 0.3 |
| pDMAEMA- | | | | | | | | | | |
| mPEG | | | | | | | | | | |

### OptiMEM Aggregation Studies

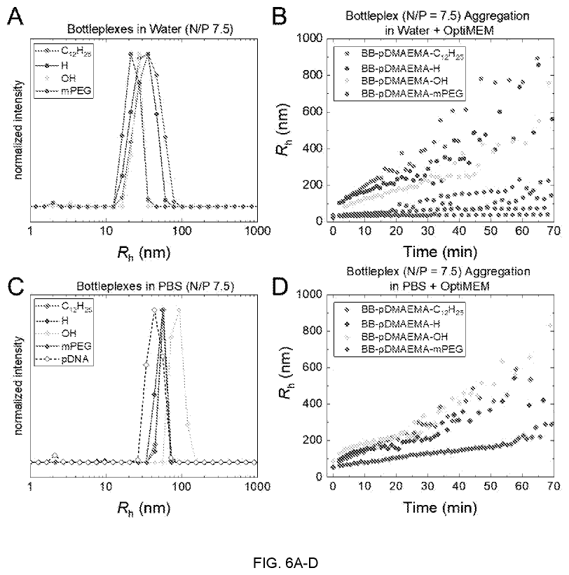

The complexes were diluted at a ratio of 2:1 OptiMEM to complex solution mixed with a pipette and immediately measured across duplicate samples as a function of time to understand the aggregation rate of each complex formulation in water and PBS. In water, the aggregation as function of time can be visualized in FIG. 6B. The rate of complex aggregation in OptiMEM follows the trend in initial complex Rh with BB-pDMAEMA-C₁₂H₂₅ aggregating most rapidly at 13 nm/min followed by BB-pDMAEMA-H (8.8 nm/min) and BB-pDMAEMA-OH (7.2 nm/min) as reported in Table 2. Interestingly, BB-pDMAEMA-mPEG did not aggregate significantly in water with the addition of OptiMEM. Over two sets of duplicate runs, one duplicate of each run did not aggregate at all as shown by the data points in FIG. 6B. The average aggregation rate was only 1.0 nm/min, an almost six-fold decrease compared to the —C₁₂H₂₅ end-group (Table 2). Without wishing to be bound to theory, the colloidal stability afforded by the pegylation of cationic complex polymers when complexed with pDNA may be indicative of increased circulation colloidal stability if these systems are to be applied in vivo and indicate better transfection of RNP cargos.

In general, the aggregation rates in PBS were less severe than in water as shown in FIG. 6D. However, BB-pDMAEMA-OH aggregated to the largest sizes over time at a rate of 9.2 nm/min from an original complex that was nearly 4× larger than the complex formed at N/P 7.5 in water (Table 2). The BB-pDMAEMA-C₁₂H₂₅ and —H complexes aggregated at a slower rate in PBS than what was observed in water, but BB-pDMAEMA-mPEG still showed the least amount of aggregation upon the addition of OptiMEM only increasing in hydrodynamic radius at a rate of 2.6 nm/min. The PEGylated complexes have increased aggregation stability over the other end-group modified analogues regardless of formulation media.

Some of the differences in aggregation behavior observed in FIG. 6A-6D can be explained by looking at the results of a dye exclusion assay (FIG. 7A and FIG. 7B). In water, all bottlebrush polymers were able to sufficiently bind pDNA at N/P ratios of 5, 7.5, and 10 evidenced by the nearly complete knockdown of fluorescence intensity by the bottlebrush exclusion of pDNA intercalating small molecule dye. Upon the addition of OptiMEM, the normalized fluorescence intensity increased indicating the unbinding of the bottlebrush from pDNA. Therefore, the aggregation behavior observed in FIG. 6B can be understood as a function of the slow unbinding of pDNA from the complex as a function of time. In contrast, the bottlebrushes show very low dye exclusion at all N/P ratios in PBS. And upon the addition of OptiMEM, there was little change in fluorescence intensity (FIG. 7B).

The solution behaviors of complexes formulated in water and PBS were distinct from one another. In water, all bottlebrushes strongly bound to pDNA and formed well defined complexes of decreasing size with increasing length of oligoPEG added to the exterior of the bottlebrush sidechains. Upon addition of OptiMEM, unbinding of pDNA and bottlebrush was evidenced by dye exclusion (FIG. 7A) and by the rapid aggregation in most samples (FIG. 6B). Interestingly, the PEGylated system did not aggregate significantly upon the addition of OptiMEM and had the slowest aggregation of all complexes. In PBS, the overall complex size was much larger, and the binding between bottlebrush and pDNA does not exclude as much dye as in water (FIG. 7B). The larger complexes aggregated at slower rates upon the addition of OptiMEM compared to those in water (FIG. 6D). In PBS, the BB-pDMAEMA-OH sample formed the largest complex and showed the most aggregation as a function of time with OptiMEM addition. Without wishing to be bound to theory, the BB-pDMAEMA-OH was hypothesized to show the best transfection in PBS based on size.

### Transfection Results

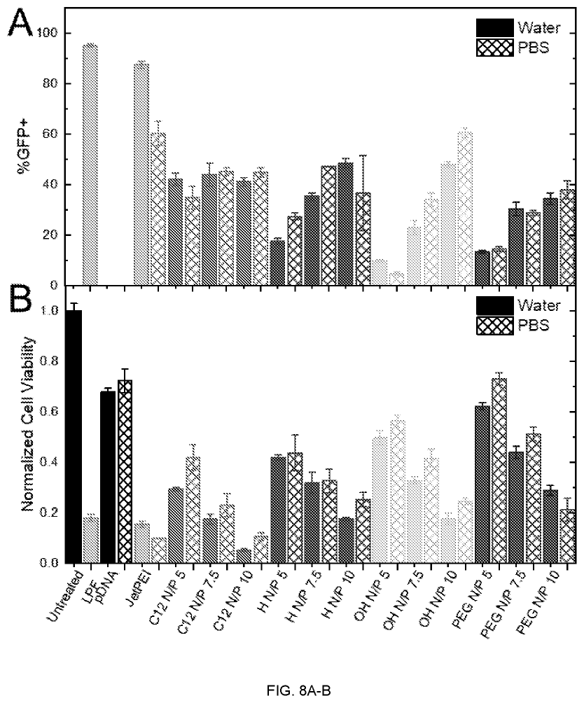

FIG. 8A reports the transfection efficiency of each complex in water and PBS at N/P ratios of 5, 7.5, and 10 compared positive controls of Lipofectamine2000 and JetPEI and negative controls of untreated cells and pDNA only. The positive controls achieved 85+% GFP+ cells when formulated in water. However, JetPEI, when formulated in PBS, showed approximately 60% GFP+ cells. The top complex transfection results were achieved by BB-pDMAEMA-OH at N/P=10 in PBS achieving a transfection of 60+% GFP+ cells. Comparing the toxicity of this formulation to JetPEI in PBS, FIG. 8B, using a CCK-8 assay showed that the BB-pDMAEMA-OH formulation in PBS was less toxic than JetPEI while achieving the same transfection efficiency. By the CCK-8 assay, the pegylated complexes that aggregated the least had the lowest amount of toxicity, but they also had the lowest % GFP+ cells transfected across N/P ratios. However, the colloidal stability still has potential as a CRISPR/Cas9 RNP delivery vector. Preliminary studies suggest that trends exist in transfection efficiency as a function of end-group chemistry where the dodecyl end-group is the most toxic to cells while the pegylated bottlebrush is the least toxic. The highest transfection efficiency was tied to stability in OptiMEM with the BB-pDMAEMA-H and BB-pDMAEMA-OH samples, which aggregated the most behind BB-pDMAEMA-C₁₂H₂₅ while being less toxic, exhibited the highest transfection efficiencies in water and PBS.

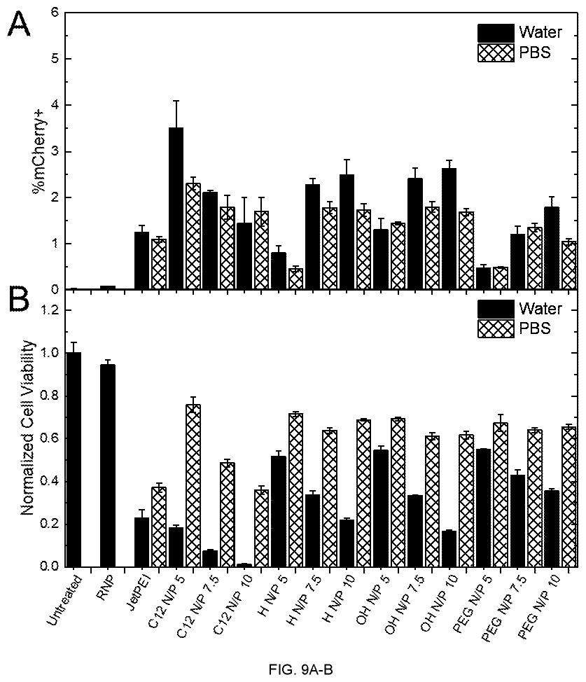

FIG. 9A reports the transfection efficiency of each complex in water and PBS at N/P ratios of 5, 7.5, and 10 compared positive controls of Lipofectamine2000 and JetPEI and negative controls of untreated cells and RNP only. The positive control achieved about 1% mCherry+ cells when formulated in water or PBS. The top complex transfection results were achieved by BB-pDMAEMA-C₁₂H₂₅ at N/P=5 in water achieving a transfection of 3.5% mCherry+ cells. Comparing the toxicity of this formulation to JetPEI in PBS using a CCK-8 assay, FIG. 9B, showed that each bottleplex was able to deliver RNP more efficiently than the JetPEI positive control with at least one formulation condition, due to the toxicity of JetPEI when delivering RNP complexes. Similar to the pDNA bottleplexes, when the BB-pDMAEMA-X family was complexed with RNP in PBS, a larger number of +mCherry cells were measured compared to formulations prepared in water due to differences in cell viability. The BB-pDMAEMA-C₁₂H₂₅ bottleplex formulated in PBS at N/P 5 achieved the highest effective expression of mCherry and displayed a 4.25-fold increase in effective expression over the JetPEI control. However, when the N/P ratio of BB-pDMAEMA-C₁₂H₂₅ increased, there was a sharp decline in viable cells expressing mCherry linked directly with the cytotoxicity, shown in FIG. 9B.

### Overall Results

The study demonstrates an advantageous expansion of the usage of bottlebrush polymer transfection agents in new cell lines, with new nucleic acid cargo, and to promote transitions from in vitro to in vivo delivery. End-group modified pDMAEMA bottlebrush polymers (BB-pDMAEMA-H, BB-pDMAEMA-OH, and BB-pDMAEMA-mPEG) were synthesized from a single template using post-polymerization modification optimized previously for acrylamide containing bottlebrush copolymers used for oral drug delivery. It is herein shown that the end-group modified bottlebrushes behave differently in formulation media between water and PBS leading to differences in polymer-pDNA binding, complex size, and aggregation behavior upon the addition of OptiMEM. The largest complex formulation of BB-pDMAEMA-OH in PBS resulted in the best transfection efficiency of GFP to HEK-293 cells at N/P=10. Like pegylated micelle complexes, the PEG outer-shell imparted colloidal stability in both water and PBS in the presence of OptiMEM. This increased colloidal stability, however, resulted in the lowest transfection efficiencies while also being the least toxic.

### Example 3

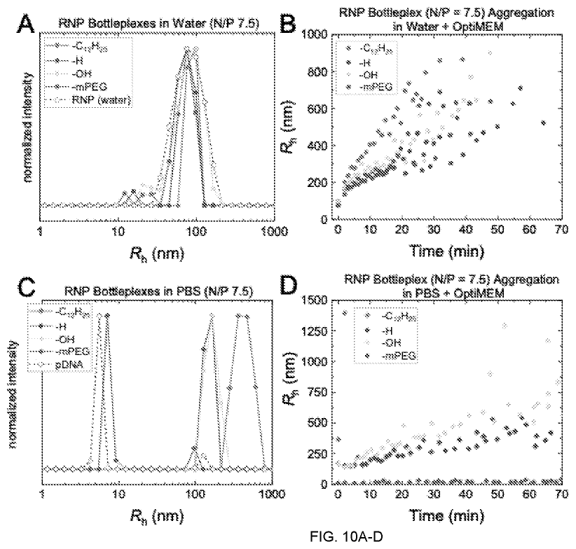

The BB-X family was further complexed with the CRISPR-Cas9 guide RNA (gRNA)-protein RNP complex to understand the size, aggregation, and binding strength. Each end-group-modified pDMAEMA bottlebrush was complexed with RNP in water and PBS. The hydrodynamic radii of the bottleplexes in water were consistent with the size of RNP only in water, suggesting that the cationic bottlebrush polymers complexes with a pre-existing aggregate of RNP yielding bottleplexes ranging in size from 75 to 100 nm in Ri, (FIG. 10A and Table 2). In contrast, the RNP alone in PBS existed as the monomodal population of distinct protein complexes (Rh=4.5 nm). Significant aggregation of the bottleplexes when RNP was complexed with BB—C12H25, —H, and —OH was observed with hydrodynamic radii greater than 150 nm (FIG. 10C and Table 2). In contrast, the PEGylated BB-mPEG formed bottleplexes with RNP in PBS with Rh equal to 7.1 nm, suggesting that these systems may exist as unimolecular PEGylated bottlebrushes complexing one or more RNP. The complexation of multiple RNP units to a single PEGylated micelleplex has been previously observed. To investigate the solution stability of these RNP bottleplexes during transfection, Opti-MEM was again added to the bottleplexes, and the aggregation rate was monitored as a function of time (70 min).

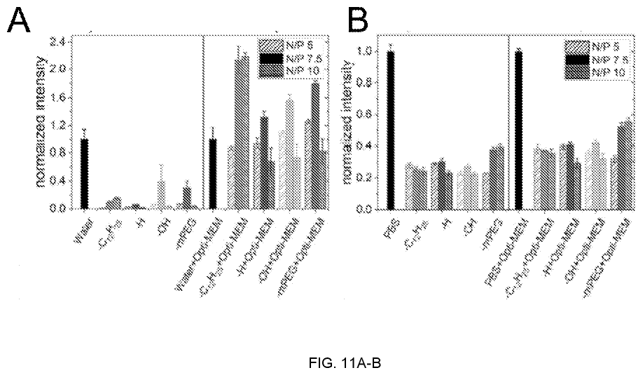

Upon the addition of Opti-MEM, the general aggregation trends followed what was observed previously with pDNA in water. The BB—C12H25 aggregated at the fastest rate, followed by BB—H and —OH while BB-mPEG underwent the least amount of aggregation. Each of the RNP formulations in water resulted in rapid aggregation at a rate estimated to be greater than 10 nm/min and almost 2× faster than the pDNA bottleplexes across all vehicles (FIG. 10B and Table 2). Based on dye exclusion, upon Opti-MEM addition, RNP bottleplexes formed in water showed complete unbinding and fluorescence values above the negative control (FIG. 11A). The severe aggregation seen by DLS and the autofluorescence of the protein structure support the concern of denaturation and are hypothesized to be detrimental to the efficacy of RNP transfection, in contrast to the advantages seen for pDNA transfection. As shown in FIG. 10D, the BB—C12H25 RNP complex in PBS aggregated so rapidly upon the addition of Opti-MEM that the size of the bottleplex aggregate was not measurable after 5 min. In contrast, the BB-mPEG bottleplex with RNP, upon the addition of Opti-MEM, remained colloidally stable for over 1 h. This behavior was unique to the PEGylated bottleplex system and alluded to potential applications of future intravenous administration. Dye exclusion of the RNP bottleplexes shows how RNP remains bound after Opti-MEM was added to the complexes formed in PBS (FIG. 11B). Overall, the data suggests that colloidal and protein stability was enhanced when bottleplexes were preformed in PBS and when PEGylated, which in turn may result in higher effective transfection as the aggregate size and binding may affect how the bottleplex composition interacts intracellularly.

Other embodiments are in the claims.

What is claimed is:

1. A polymer of formula (I) 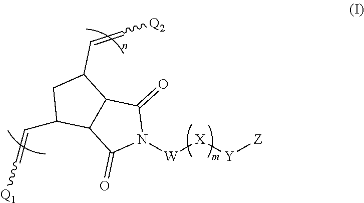
wherein
W is alkyl alkanoate;
X is a monomeric unit of aminoalkyl acrylate;
Y is a bond or S;
Z is hydrogen, alkyl, 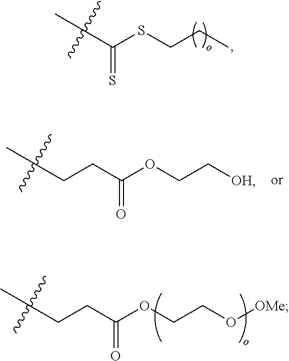
Q₁ is hydrogen or an end group;
Q₂ is hydrogen or an end group;
n is from 1 to 1000;
m is from 1 to 1000; and
o is from 1 to 20;
or an ion or salt thereof.

2. The polymer of claim 1, wherein the aminoalkyl acrylate is 2-(dimethylamino)ethyl methacrylate (DMAEMA).

3. The polymer of claim 1 or 2, wherein n is from 10 to 50.

4. The polymer of claim 3, wherein n is about 30.

5. The polymer of any one of claims 1 to 4, wherein m is from 10 to 50.

6. The polymer of claim 5, wherein m is about 20.

7. The polymer of any one of claims 1 to 6, wherein Q₁ or Q₂ is an end group selected from the group consisting of alkyl, aryl, and heterocyclyl.

8. The polymer of claim 7, wherein Q₁ is phenyl.

9. The polymer of any one of claims 1 to 8, wherein Q₂ is hydrogen.

10. A complex comprising the polymer of any one of claims 1 to 9 and a negatively charged biological agent.

11. The complex of claim 10, wherein the negatively charged biological agent comprises a nucleic acid.

12. The complex of claim 11, wherein the nucleic acid comprises DNA or RNA.

13. The complex of claim 10, wherein the nucleic acid comprises gRNA, mRNA, tmRNA, tRNA, rRNA, siRNA, shRNA, PNA, ssRNA, dsRNA, pDNA, ssDNA, dsDNA, a DNA: RNA hybrid molecule, a plasmid, an artificial chromosome, cDNA, a PCR product, a restriction fragment, a ribozyme, an antisense construct, or a combination thereof.

14. The complex of claim 10, wherein the negatively charged biological agent comprises a protein.

15. The complex of claim 14, wherein the protein comprises a ribonucleoprotein.

16. The complex of claim 15, wherein the ribonucleoprotein comprises a virus, a ribosome, telomerase, Ribonuclease P (RNase P), a heterogeneous ribonucleoprotein particle (hnRNP), or a small nuclear ribonucleoprotein particle (snRNP).

17. The complex of claim 14, wherein the protein comprises a nuclease.

18. The complex of claim 17, wherein the nuclease comprises a zinc finger nuclease (ZFNs), a transcription-activator like effector nucleases (TALEN), or a Cas protein.

19. The complex of claim 18, wherein the Cas protein comprises Cas2, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas12, Cas13, Cas14, CfpI, CasI, CasIB, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, modified versions thereof, or combinations thereof.

20. The complex of claim 18, wherein the Cas protein is Cas9.

21. The complex of claim 10, wherein the negatively charged biological agent comprises a nucleic acid and a nuclease.

22. The complex of claim 21, wherein the negatively charged biological agent comprises gRNA and a Cas protein.

23. The complex of any one of claims 10 to 22, wherein the negatively charged biological agent is bound noncovalently to the polymer.

24. The complex of any one of claims 10 to 22, wherein the polymer is complexed with the negatively charged biological agent.

25. A composition comprising the complex of any one of claims 10 to 24 and a liquid carrier.

26. A method comprising contacting a cell with the complex of any one of claims 10 to 24, wherein the biological agent is delivered into the cell.

27. A compound of formula (II) 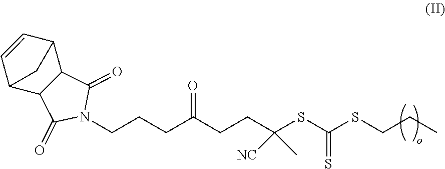
wherein
is from 1 to 20.

28. A polymer of formula (III) 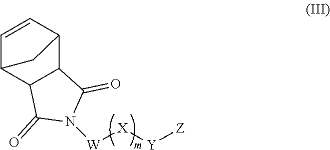
wherein
W is alkyl alkanoate;
X is a monomeric unit of aminoalkyl acrylate;
Y is a bond or S;
Z is hydrogen, alkyl, 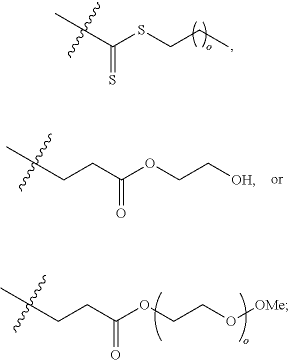
m is from 1 to 1000; and
o is from 1 to 20;
or an ion or salt thereof.
